(19) United States
(12) Patent Application Publication
(10) Pub. No.: US 2024/0058142 A1
(43) Pub. Date: Feb. 22, 2024

(54) 3D PRINTED DEVICES AND METHOD OF USE

(71) Applicant: NTT RESEARCH, INC., Sunnyvale, CA (US)

(72) Inventors: Bernhard WOLFRUM, Sunnyvale, CA (US); Tetsuhiko TESHIMA, Sunnyvale, CA (US); Lukas HIENDLEMEIER, Sunnyvale, CA (US); Korkut TERKAN, Sunnyvale, CA (US)

(73) Assignee: NTT RESEARCH, INC., Sunnyvale, CA (US)

(21) Appl. No.: 18/266,996

(22) PCT Filed: Dec. 14, 2021
(86) PCT No.: PCT/US2021/063254

§ 371 Date: Jun. 13, 2023

Related U.S. Application Data
(60) Provisional application No. 63/125,207, filed on Dec. 14, 2020.

(51) Int. Cl. A61F 2/844; B33Y 80/00; B33Y 70/10

(52) U.S. Cl. CPC A61F 2/844; B33Y 80/00; B33Y 70/10; B29K 2995/0094; B29K 2075/00

(57) ABSTRACT

A method for manufacturing single unit structures with three dimensional printing and various materials formable into three dimensional assemblies that may be used for various purposes.

### PRIORITY/RELATED APPLICATIONS

This application claims priority and the benefit under 35 USC 119(e) to U.S. Provisional Patent Application Ser. No. 63/125,207, filed Dec. 14, 2020 and entitled “3D Printed Devices and Method of Use”.

### FIELD

The disclosure relates generally to microscale or nanoscale fabricated devices and in particular to the assembly to 2D planar structures into three dimensional structures.

### BACKGROUND

Technologies exist today that allow 2D planar shapes to the assembled into 3D microscale shapes. These 3D microscale shapes maybe used for various applications, including foldable electronics, piezeoelectric systems/MEMs, implantable medical devices or devices, such as a stent, biosensors, wireless devices and/or photodetectors/batteries for example. To date these 3D microscale shapes are manufactured using surface-tension based assembly, residual stress driven actuation, electric, magnetic and thermal activation and/or shape memory alloy activation. However, these known techniques and structures have limitations in applicable materials for these techniques. Furthermore, these known techniques lack the ability to achieve 3D geometries in a deterministic manner. In addition, these known techniques do not allow the 3D geometries to have significant changes in dimensionality (individual elements that shift, are inclined or otherwise move relative to each other) thus limiting the devices that can be made using these techniques. The flexible and multi-dimensional shape change leads to a wide variety of application including biomedical devices such as transformable stents (“Self-deployable origami stent grafts as a biomedical application of Ni-rich TiNi shape memory alloy foil”, Materials Science and Engineering: A, Volume 419, 2006, Pages 131-137)

Thus, it is desirable to provide a single fiber shaped unit and the assembly of those single fiber shaped unit into devices that have the desirable changes of dimensionality not provided by the known techniques and it is to this end that the disclosure is directed. In addition to the change of dimensionality, it is highly desirable to add the function or structures to fix and hold the objective devices in the final geometry after the transformation process.

### BRIEF DESCRIPTION OF THE DRAWINGS

FIG. 1 is a first embodiment of a single fiber-shaped unit;

FIG. 2 is a second embodiment of a single fiber-shaped unit;

FIG. 3 is a third embodiment of a single fiber-shaped unit;

FIG. 4 is a fourth embodiment of a single fiber-shaped unit;

FIG. 5 is a fifth embodiment of a single fiber-shaped unit;

FIG. 6 is a sixth embodiment of a single fiber-shaped unit;

FIG. 7 is a seventh embodiment of a single fiber-shaped unit;

FIG. 8 is an eighth embodiment of a single fiber-shaped unit;

FIG. 9 illustrates an example of a commercially available three dimensional printer that may be used to manufacture the units 10-180 shown in FIGS. 1-8;

FIG. 10 illustrates a set of dimensions for the rectangular ring unit 170 shown in FIG. 7;

FIGS. 11A and 11B illustrate the dimensional changes that can be performed using the units in FIGS. 1-8 with the rectangular ring being used as an example;

FIG. 12 illustrates an example of one or more units being assembled to be aligned in a row;

FIG. 13 illustrates an example of one of more units being assembled in a pair in parallel configuration;

FIG. 14 illustrates an example of one of more units being assembled in a joined end configuration of two serial configurations;

FIG. 15 illustrates an example of one of more units being assembled in a circuit configuration;

FIG. 16 illustrates an example of an assembly of units with a radius of curvature;

FIGS. 17A and 17B illustrates a layer of curved assembled units forming a tube structure and a plurality of layers of curved assembled units forming a longer tube structure, respectively;

FIG. 18 illustrates an example of an assembly of units forming an ring structure;

FIG. 19 illustrates an example of an assembly of units forming a caged structure;

FIG. 20 illustrates an example of an assembly of units folded and being stretched to form a caged structure;

FIG. 21 illustrates an example of an assembly of units forming a rhomboidal shape circuit;

FIG. 22 illustrates examples of an assembly of units forming other circuits; and

FIG. 23 illustrates examples of medical devices that may be formed using an assembly of single fiber units.

### DETAILED DESCRIPTION OF ONE OR MORE EMBODIMENTS

The disclosure is particularly applicable to a single fiber unit and its assembly into devices with dimensionality changes that is made from photo-curable material using stereolithographic 3D printing for medical devices and it is in this context that the disclosure will be described. It will be emphasized, however, that the single units and assemblies in accordance with the disclosure has greater utility. For example, the devices and assemblies can be made of other materials and manufacturing techniques that are within the scope of the disclosure. Furthermore, the devices and assemblies disclosed may be used for different use cases in addition to the medical device use case disclosed below.

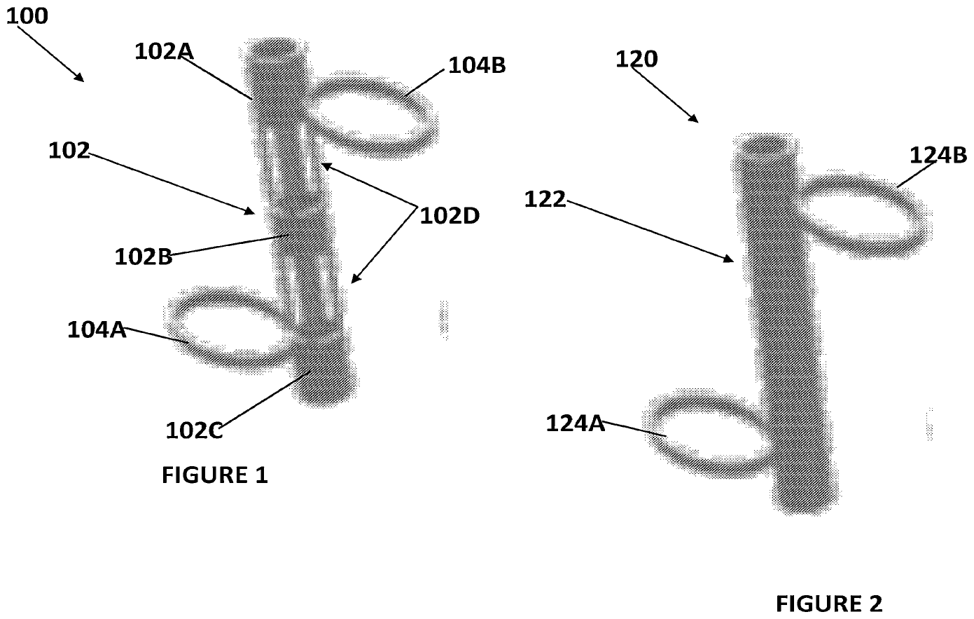

FIG. 1 is a first embodiment of a single fiber-shaped unit 100. The single unit 100 may have a central structure 102, that may be a cylindrical shaped, that has a length and one or more attachment mechanisms 104, such as loops or strings, that are attached to the central structure 102. In this embodiment, a first attachment 104A is attached to one end of a first side of the central structure 102 and a second attachment 104B is attached to an opposite end of a second side of the central structure 102 as shown in FIG. 1. In the embodiment in FIG. 1, each attachment 104 may have a closed circular loop shape. The single unit 100 in FIG. 1 may have a grid pattern shape. In this embodiment, the central structure 102 has a solid first end portion 102A, a middle solid portion 102B and a solid second end portion 102C in which these portions 102A-102C are connected by a portion 102D that has a solid center and one or more legs that connect the other portions to each other. The hollow structures of the central structures 102 helps the users not only to reduce the amount of weight of the whole system, but also to embed or integrate the other chemicals or structures inside the central structures, for example the drug-containing hydrogels for the drug release functions.

FIG. 2 is a second embodiment of a single fiber-shaped unit 120 that may have a central structure 122, that may be a cylindrical shaped and hollow and may have a length and one or more attachment mechanisms 124, such as loops or strings, that are attached to the central structure 122. In this embodiment, a first attachment 124A is attached at one end of a first side of the central structure 122 and a second attachment 124B is attached at an opposite end of a second side of the central structure 122 as shown in FIG. 2. In the embodiment in FIG. 2, each attachment 104 may have a closed circular loop shape. The single unit 120 in FIG. 2 may have a hollow cylinder shape. The hollow cylindrical shape in the central structure enables not only to reduce the amount of chemicals that are necessary for printing, but also increase the geometrical moment of inertia and making them flexible and at the same time hard to be bent and without being broken.

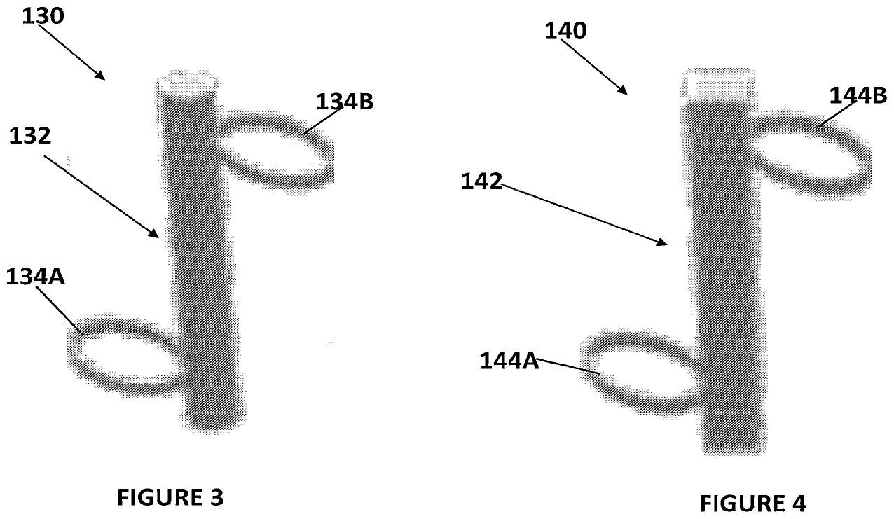

FIG. 3 is a third embodiment of a single fiber-shaped unit 130 that may have a central structure 132, that may be cylindrically shaped and solid and may have a length and one or more attachment mechanisms 134, such as loops or strings, that are attached to the central structure 132. In this embodiment, a first attachment 134A is attached tot one end of a first side of the central structure 132 and a second attachment 134B is attached to an opposite end of a second side of the central structure 132 as shown in FIG. 3. In the embodiment in FIG. 3, each attachment 134 may have a closed circular loop shape.

FIG. 4 is a fourth embodiment of a single fiber-shaped unit 140 that may be a polygonal prism. The unit 140 may have a central structure 142, that may have a rectangular or square cross section and be solid and may have a length and one or more attachment mechanisms 144, such as loops or strings, that are attached to the central structure 142. In this embodiment, a first attachment 144A is attached to one end of a first side of the central structure 142 and a second attachment 144B is attached to an opposite end of a second side of the central structure 142 as shown in FIG. 4. In the embodiment in FIG. 4, each attachment 144 may have a closed circular loop shape.

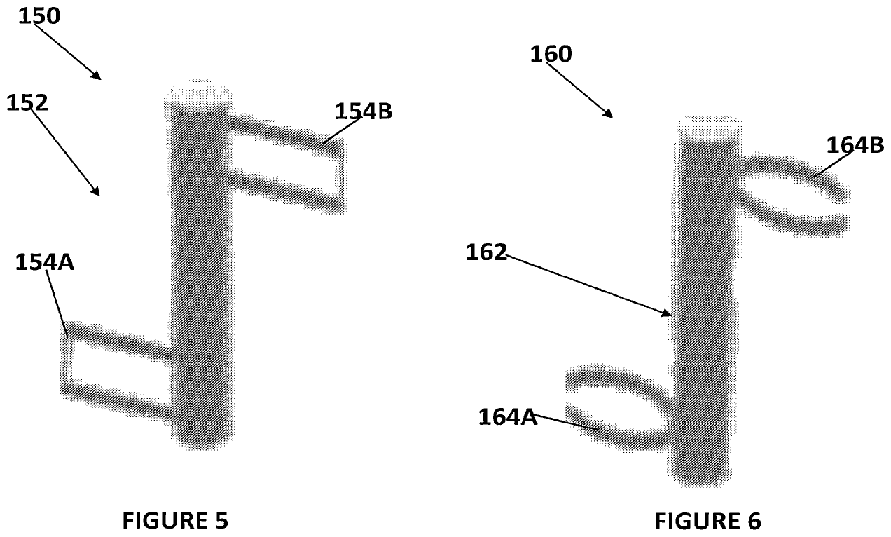

FIG. 5 is a fifth embodiment of a single fiber-shaped unit 150 that may be polygonal ring. The unit 150 may have a central structure 152, that may be cylindrically shaped and solid and may have a length and one or more attachment mechanisms 154, such as loops or strings, that are attached to the central structure 152. In this embodiment, a first attachment 154A is attached to one end of a first side of the central structure 152 and a second attachment 154B is attached to an opposite end of a second side of the central structure 152 as shown in FIG. 5. In the embodiment in FIG. 5, each attachment 154 may have a closed square or rectangular shape.

FIG. 6 is a sixth embodiment of a single fiber-shaped unit 160 that may be reversable coupler. This unit 160 may have a central structure 162, that may be solid and cylindrically shaped and may have a length and one or more attachment mechanisms 164, such as loops or strings, that are attached to the central structure 162. In this embodiment, a first attachment 164A is attached to one end of a first side of the central structure 162 and a second attachment 164B is attached to an opposite end of a second side of the central structure 162 as shown in FIG. 6. In the embodiment in FIG. 6, each attachment 164 may have an open circular shape that has an opening at an end opposite from the end connected to the central structure.

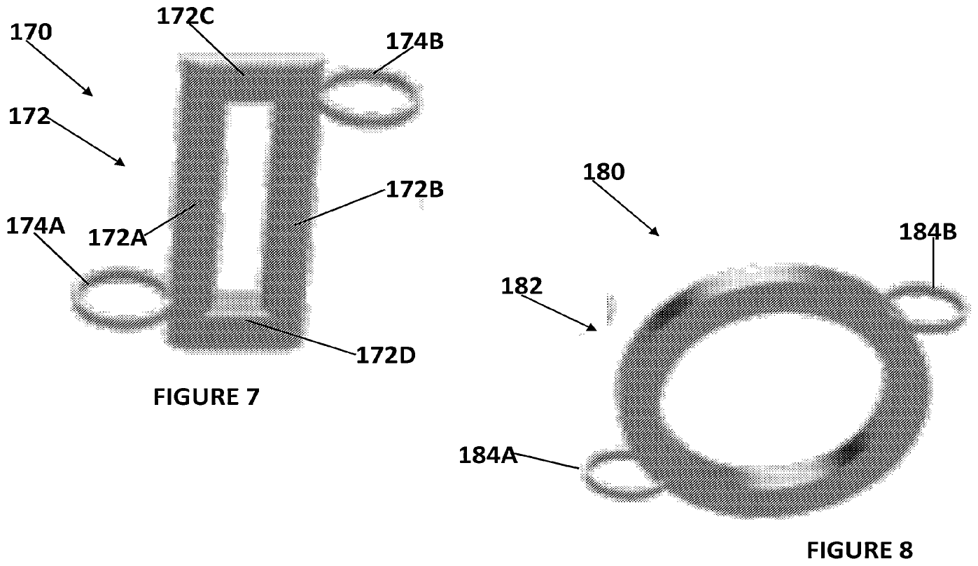

FIG. 7 is a seventh embodiment of a single fiber-shaped unit 170 that may be a rectangular ring. The unit 170 that may have a central structure 172, that may be a rectangular shape. The central structure 172 may a first cylindrical post 172A, and a second cylindrical post 172B spaced apart from each other by a top portion 172C and a bottom portion 172D (all of which are solid) so that the unit 170 has a open middle portion as shown in FIG. 7. The unit 170 may also have one or more attachment mechanisms 174, such as loops or strings, that are attached to the central structure 172. In this embodiment, a first attachment 174A is attached at one end of the first post 172A of the central structure 172 and a second attachment 174B is attached at an opposite end of the second post 172B of the central structure 172 as shown in FIG. 7. In the embodiment in FIG. 7, each attachment 134 may have a closed circular loop shape.

FIG. 8 is an eighth embodiment of a single fiber-shaped unit 180 that is a circle ring. The unit 180 may have a central structure 182, that may be a solid circular ring shape with flat sides. The unit 180 may have one or more attachment mechanisms 184, such as loops or strings, that are attached to the central structure 182. In this embodiment, a first attachment 184A is attached to a first part of the central structure 182 and a second attachment 184B is attached to a second side of the ring of the central structure 182 as shown in FIG. 8. In the embodiment in FIG. 8, each attachment 184 may have a closed loop shape.

The positioning of the attachments 104A-184A, 104B-184B on each central structure 102-182 on opposite sides of the central structure allows the single units to be assembled and to have the desirable change in dimensionality as discussed below in more detail. Furthermore, as shown in FIGS. 1-8 the central structure can be any of the shapes shown as well as other shapes. In addition, the attachments 104-184 may be any of the shapes shown in FIGS. 1-8 as well as other shapes.

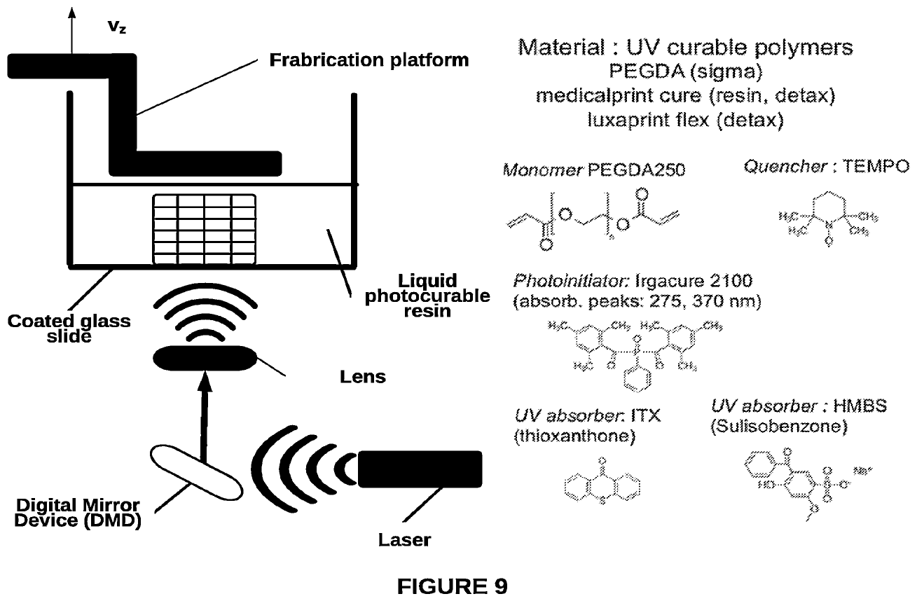

Each of the above embodiments of the single fiber shaped units 100-180 may be manufactured using various known or yet to be developed manufacturing techniques. For example, in one embodiment, each of the units 100-180 may be manufactured using stereolithography (SLA) 3D printing with a digital light projector (DLP) with a constrained-surface/bat configuration as shown in FIG. 9. For example, the units 100-180 may be manufactured using a commercially available MiiCraft Ultra 50 3D printer (further details may be found at //miicraft.com/product-2/that is incorporated herein by reference. Using the 3D printer, one can successively print in a layer-by-layer manner and objects are built by spatially controlled photopolymerization. Each unit may be created by hanging from the bat suspended above the resin reservoir shown in FIG. 9 and a light source is located underneath the tank as shown in FIG. 9. In this example, the material used for each unit 100-80 may be a UV-curable polymer and the other materials as shown in FIG. 9.

Alternatively, the single units in FIGS. 1-8 may be manufactured out of any material that is compatible meaning a material that can be 3D printed into the single fiber units shown in FIGS. 1-8. For example, the materials for each single fiber may be a photo-curable polymer, such as epoxy resin, acrylic resin, acrylonitrile butadiene styrene (ABS), gypsum hemihydrate, polylactic acid (PLA) resin, nylon resin, silicone rubber, polyurethane based shape memory polymers, and printable metals such as iron, maraging steel, stainless, aluminum, copper, titanium, chromoly molybdenum steel, and inconel. For example, the polymeric materials for each single fiber may be composite with the other functional chemicals such as a polymer/micro-nanoparticle composite, a mixture of ceramic powders, a mixture of conductive polymers, such as polyacetylene, polyparaphenylene, polyaniline, polythiophene, poly paraphenylene vinylene, and polypyrrole, a mixture of high-aspect-ratio materials such as carbon nanotubes or graphene, or transition metal dichalcogenide monolayers, and/or drug embedded photocurable polymers when used for a drug delivery system. In general, the material used may be dependent of the use case of the single fiber units since they may have conductive particles when being used as electrodes or the drug embedded polymers for a drug delivery system.

Alternatively, instead of the stereolithographic 3D printing method using the UV curable polymers set forth above, the single fiber units may be manufactured using other types of 2D/3D printers such as inkjet printing, selecting laser sintering and/or fused filament deposition for example. In accordance with the disclosure, any printing method that can generate sub-micron scale devices with a width of a loop/string I_{D} of the single fiber unit being between 500 nm to 10 cm and a height, H, between the loop/string on each side of the single fiber unit being between 1 μm to 50 cm.

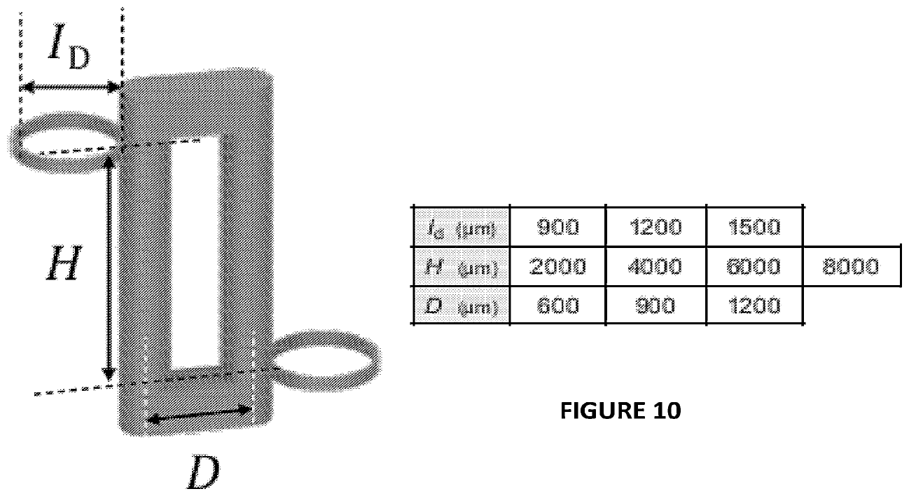

FIG. 10 illustrates a set of dimensions for the rectangular ring unit 170 shown in FIG. 7. These dimensions affect how one or more units may be assembled into 3D structures. The dimensions may include an attachment diameter dimension (I_{D}), a height, H, between the first and second attachment and a distance, D, between a center of the first and second posts of the center structure. All of the dimensions are measured in micrometers (μm). The transformability of a series of the units (how they are sliding or bent) is controllable by changing the length and thickness of the central structures and the shapes of attachment parts, especially the attachment diameter dimension. The units with all of the shown indicated parameters were 3D-printed in the direction along the long axis of central structures. The similar dimensions to the single post central structures can be 3D-printed in the units shown in from FIG. 1 to FIG. 7. Similar dimensions to the single attachment structures can be applicable to the units shown in from FIG. 1 to FIG. 8.

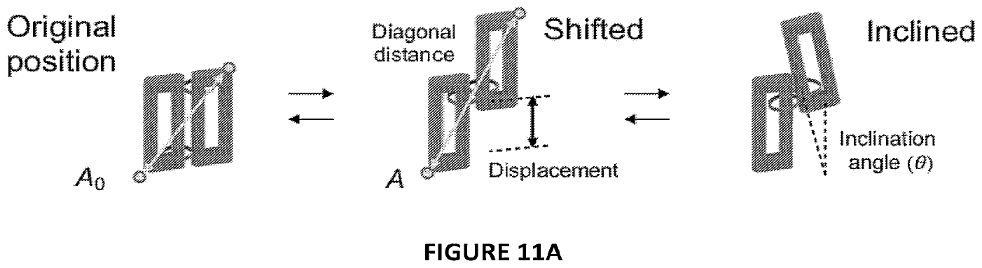

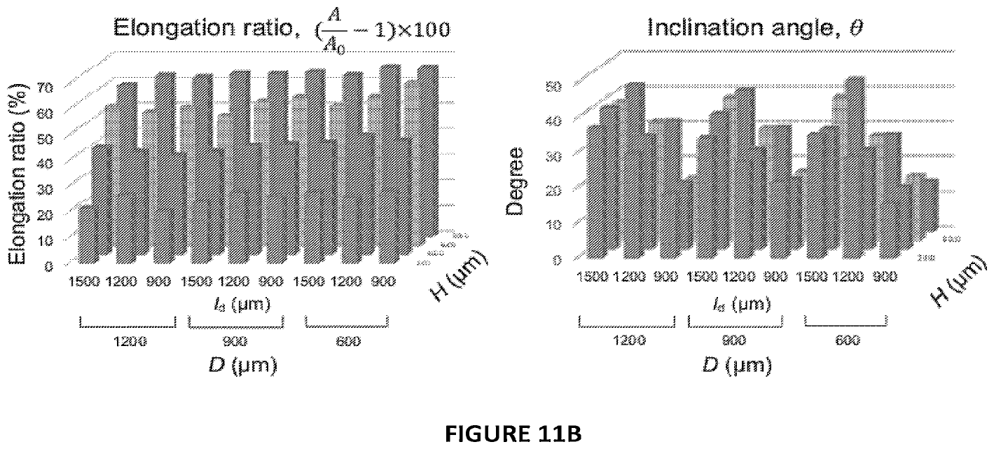

FIGS. 11A and 11B illustrate the dimensional changes that can be performed using the units in FIGS. 1-8 with the rectangular ring being used as an example. As shown in FIG. 11, two original rectangular ring single units are connected to each other and have a diagonal distance A₀ from a lower corner of the first unit to an opposite top corner of the second unit. The dimensional changes that can occur include shifting and/or inclination that may each occur alone or in any combination. With shifting, the diagonal distance, A, increases and there is a displacement between a bottom portion of each single unit. With inclination, there is an inclination angle θ between the two single units as shown in FIG. 11A. FIG. 11B shows the ranges of elongation ratios [(A/A₀−1)×100] as a percentage for different dimensions (D, I_{D} and H in μm) of each single unit wherein the ratio is from about 20% to about 70+%. FIG. 11B also shows the inclination angle θ for different dimensions (D, I_{D} and H in μm) of each single unit wherein the inclination angle ranges from about 10° to about 50°. The charts in FIG. 11B show that the dimensional change (deformability) is controlled by the height H (with maximum elongation of 65%), and another dimensional change (bendability) is controlled by the shape/diameter I_{D} of the strings (with a maximum inclination of 42°).

Assembly of Single Units into 3D Structures

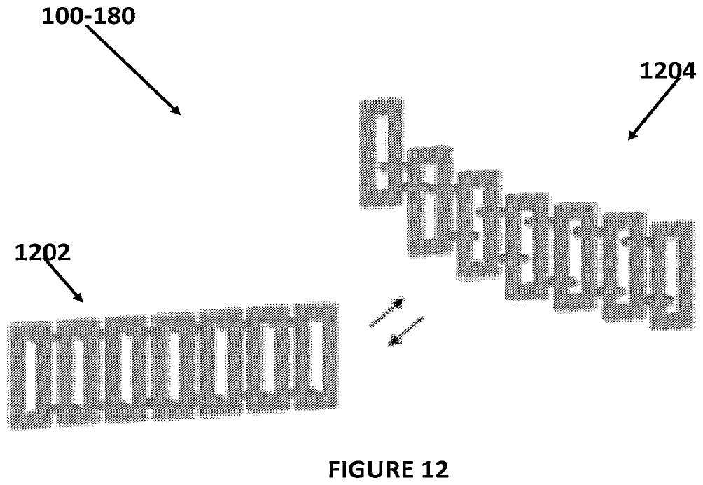

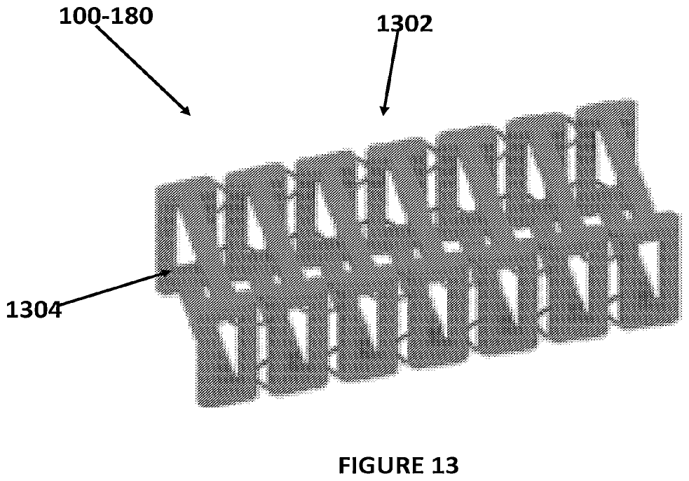

FIGS. 12-16 illustrate some examples of how the single fiber units discloses above may be used to form different three dimensional structures by assembling the single fiber units in different ways. For example, FIG. 12 illustrates two examples of one or more units 100, 180 being assembled in a line. A first assembly 1202 contains a plurality of single fiber units in a straight line while a second assembly 1204 shows the same plurality of single fiber units is a curved line (arcuate shape) as a result of shifting of different single fiber units a different distance relative to each other. FIG. 13 illustrates an example of one of more units 100-180 being assembled in a parallel configuration 1302. In this assembly, each unit 100-180 may be connected to its opposite unit by a board portion 1304 that spans between an open region between the posts of the first unit and the unit opposite as shown in FIG. 13 on both ends of each single unit. The board portions 1304 may provide addition strength to the assembly 1302. The assembly 1302 is two equal length strings of single fiber units spaced apart in a parallel configuration with the boards 1304 connecting the respective pair of single fiber units.

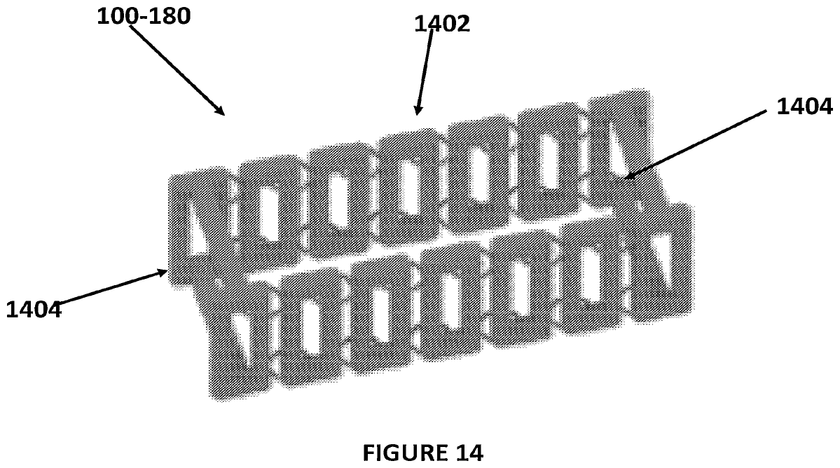

FIG. 14 illustrates an example of one of more units 100-180 being assembled in a joined end configuration of two serial configurations 1402. In this assembly, each end unit in the line may be connected to its opposite unit by a board portion 1404 that spans between an open region between the posts of the first unit and the unit opposite as shown in FIG. 14. The board portions 1404 may provide addition strength to the assembly 1402. The assembly 1402 is two equal length strings of single fiber units spaced apart in a parallel configuration with the boards 1404 connected the respective pair of single fiber units at each end of the assembly.

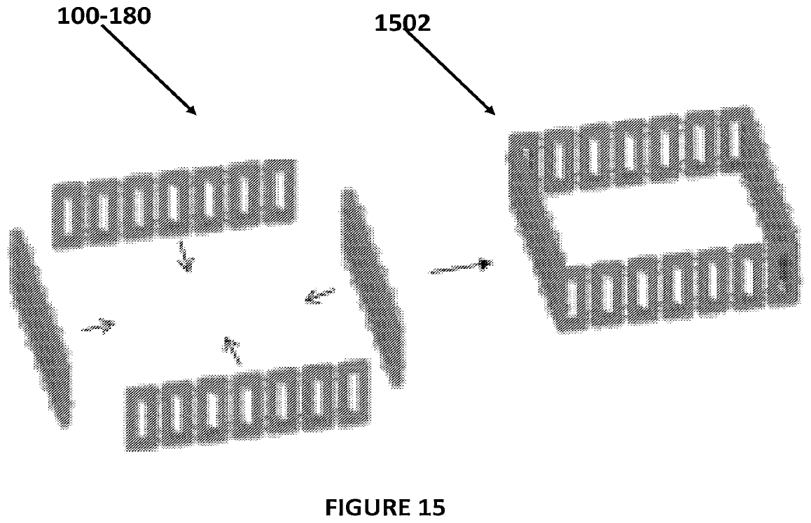

FIG. 15 illustrates an example of one of more units being assembled in a circuit configuration 1502. This assembly 1502 may be built from, in the example in FIG. 15, four equal length strings of single fiber units that are connected together at the end of each string to the end of the next string. In this example, a square circuit is assembled. If changing the length of the strings or their number, complex shapes of polygons can be assembled.

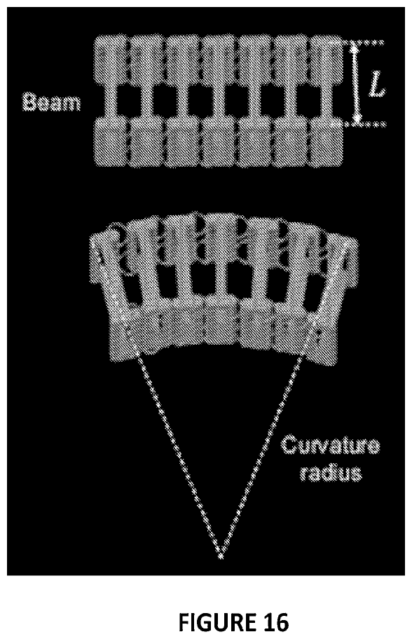

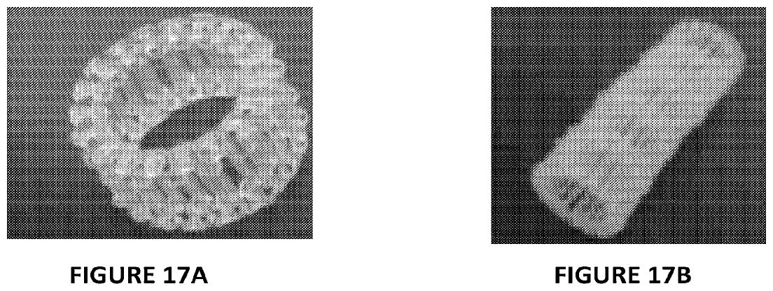

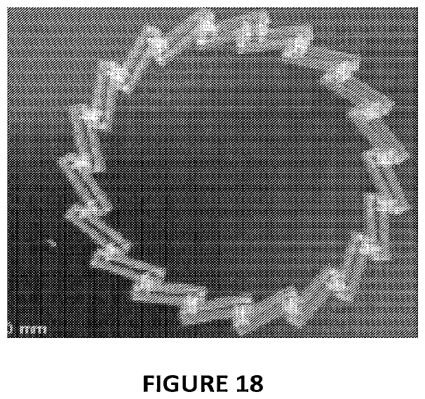

FIG. 16 illustrates an example of an assembly of single fiber units with a radius of curvature. The radius of curvature is formed because an end of unit of one side of the closer to each other and the other end of each unit a farther away (within the dimensional changes permitted by the dimensions of the loops/strings) as shown in FIG. 16. As can be seen from the above examples, the single fiber units may be assembled into various basic 3D structures that may then be assembled into devices for various uses. One such exemplary use is for various medical devices as described below in more detail. Furthermore, FIGS. 17A and 17B illustrates a layer of curved assembled units (like those shown in FIG. 16) forming a tube structure and a plurality of layers of curved assembled units forming a longer tube structure, respectively. As shown in FIGS. 17A and 17B, the hooks are fabricated at the edges of both final units in a series of units. The structure of the added hooks is partially open loop attachment, which results in the reversible and detachable connection with the final units). These tube structure can be used to form various 3D assemblies including pipes, wires, catheters, etc. FIG. 18 illustrates an example of an assembly of units forming a ring structure that can also be generated using the single fiber units. This ring structure in FIG. 18 may be used as, for example, a gear in a larger assembly. For example ring assemblies in FIGS. 17A, 17B and 18, the assembly of single fiber units may have hooks at the edges of the assembly that allow the ends to be connected to form the ring-shaped structure. The ring shapes shown have high flexibility and mechanical stability and behaves elastically when deformed due to its structure. In another example, the ring shapes can be assembled, aligned in a row to form a tubular like structure, such as a stent.

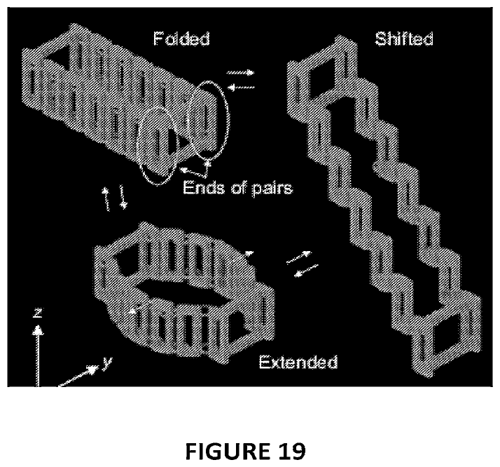

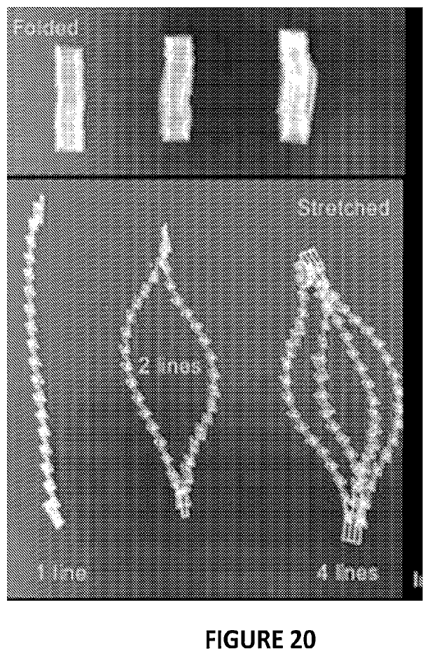

FIG. 19 illustrates an example of an assembly of units forming a caged structure and FIG. 20 illustrates an example of an assembly of units folded and being stretched to form a caged structure. As shown in FIG. 19, a caged structure can be moved into various shapes and thus has change dimensionality. For example, the assembly may have a folded state, an extended state and/or a shifted state that are all based on the single 3D assembly built using the sing fiber units. As shown in FIG. 20, the single units may be assembled into a cage structure that may be, for example, a single line of single units as shown, two lines of units connected at each end to each other and/or four lines of units connected together at each end. FIG. 20 shows those assemblies in a folded position (such as for introduction into the space) and in the stretched state when they are installed for their intended purpose. These caged structures in FIG. 20 may be used as a medical mesh.

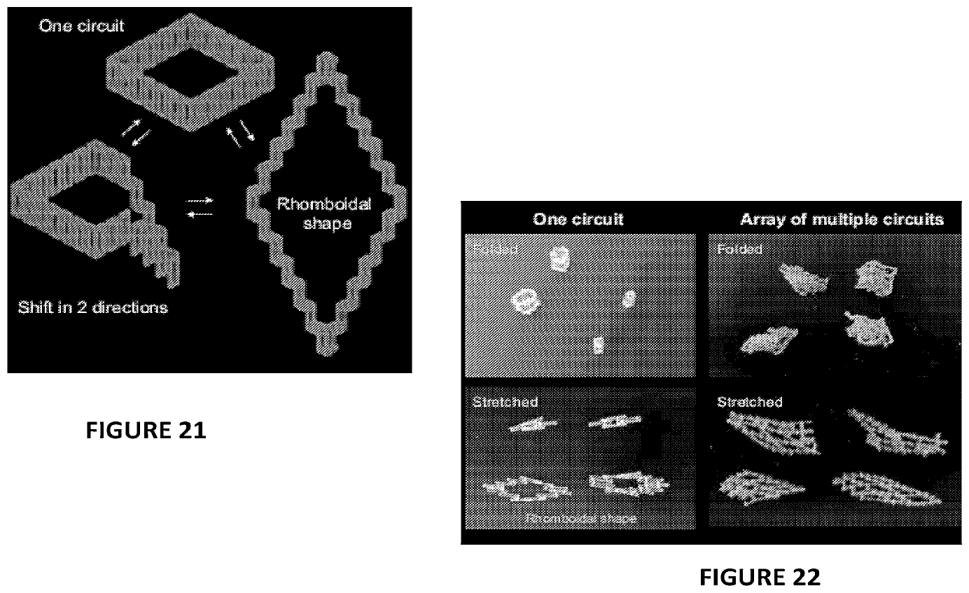

FIG. 21 illustrates an example of an assembly of units forming a rhomboidal shape circuit and FIG. 22 illustrates examples of an assembly of units forming other circuits. As shown in these figures, one circuit and/or an array of multiple circuits may be formed using assembly of the single units. For example, FIG. 21 shows a rhomboid circuit that can be shifted in two directions and forms a rhomboidal shape while FIG. 22 shows these circuits in a folded state and a stretched state. These circuits may be used to wrap around tissue. The mechanical stability of the circuits is controllable based on a number of single units in one circuit with a circuit with fewer single units being more rigid.

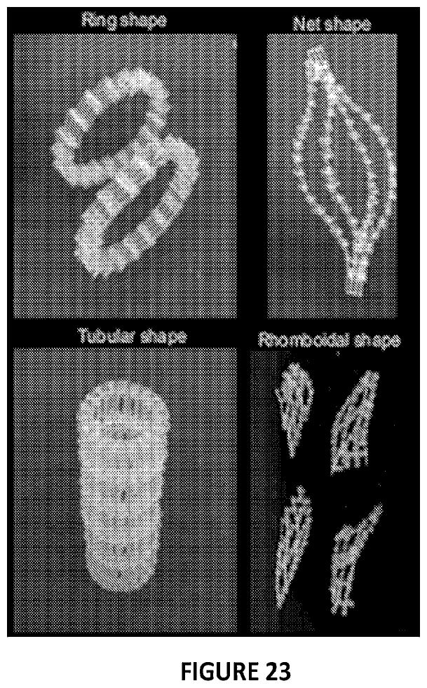

FIG. 23 illustrates examples of medical devices that may be formed using an assembly of single fiber units into ring shapes, net shapes, tubular shapes and rhomboidal shapes. These 3D assemblies created by multiple single units may be formed, for medical purposes, into a stent that may be used to treat stenosis for an abnormality of the bile duct (caused by various congenital problems, cancer or other diseases). The stent can be introduced/injected endoscopically since it can be folded and then stretched as discussed above. The stent may also be used for a tracheal stent, an esophageal stent and/or a gastrointestinal (GI) or urinary tract stent.

The shapes shown in FIG. 23 may also be used as outer rings to cover the tissue. For example, the 3D assembly may prevent hypertrophy or atrioventricular rings. The shapes may also be used for implantable net and mesh. The implantable net or mesh may be used, for example, for heart net electrodes to treat distention myocardiopathy or as surgical mesh for hernia repair. The shapes may also be used to form catheter-inducible neurosurgery tools. For example, the shapes may be used to treat cerebral aneurysm by promoting thrombus formation to prevent rupture (instead of typical coil embolization wires) and/or for inferior vena cava (IVC) filters.

The foregoing description, for purpose of explanation, has been with reference to specific embodiments. However, the illustrative discussions above are not intended to be exhaustive or to limit the disclosure to the precise forms disclosed. Many modifications and variations are possible in view of the above teachings. The embodiments were chosen and described in order to best explain the principles of the disclosure and its practical applications, to thereby enable others skilled in the art to best utilize the disclosure and various embodiments with various modifications as are suited to the particular use contemplated.

The system and method disclosed herein may be implemented via one or more components, systems, servers, appliances, other subcomponents, or distributed between such elements. When implemented as a system, such systems may include and/or involve, inter alia, components such as software modules, general-purpose CPU, RAM, etc. found in general-purpose computers. In implementations where the innovations reside on a server, such a server may include or involve components such as CPU, RAM, etc., such as those found in general-purpose computers.

Additionally, the system and method herein may be achieved via implementations with disparate or entirely different software, hardware and/or firmware components, beyond that set forth above. With regard to such other components (e.g., software, processing components, etc.) and/or computer-readable media associated with or embodying the present inventions, for example, aspects of the innovations herein may be implemented consistent with numerous general purpose or special purpose computing systems or configurations. Various exemplary computing systems, environments, and/or configurations that may be suitable for use with the innovations herein may include, but are not limited to: software or other components within or embodied on personal computers, servers or server computing devices such as routing/connectivity components, hand-held or laptop devices, multiprocessor systems, microprocessor-based systems, set top boxes, consumer electronic devices, network PCs, other existing computer platforms, distributed computing environments that include one or more of the above systems or devices, etc.

In some instances, aspects of the system and method may be achieved via or performed by logic and/or logic instructions including program modules, executed in association with such components or circuitry, for example. In general, program modules may include routines, programs, objects, components, data structures, etc. that perform particular tasks or implement particular instructions herein. The inventions may also be practiced in the context of distributed software, computer, or circuit settings where circuitry is connected via communication buses, circuitry or links. In distributed settings, control/instructions may occur from both local and remote computer storage media including memory storage devices.

The software, circuitry and components herein may also include and/or utilize one or more type of computer readable media. Computer readable media can be any available media that is resident on, associable with, or can be accessed by such circuits and/or computing components. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic tape, magnetic disk storage or other magnetic storage devices, or any other medium which can be used to store the desired information and can accessed by computing component. Communication media may comprise computer readable instructions, data structures, program modules and/or other components. Further, communication media may include wired media such as a wired network or direct-wired connection, however no media of any such type herein includes transitory media. Combinations of the any of the above are also included within the scope of computer readable media.

In the present description, the terms component, module, device, etc. may refer to any type of logical or functional software elements, circuits, blocks and/or processes that may be implemented in a variety of ways. For example, the functions of various circuits and/or blocks can be combined with one another into any other number of modules. Each module may even be implemented as a software program stored on a tangible memory (e.g., random access memory, read only memory, CD-ROM memory, hard disk drive, etc.) to be read by a central processing unit to implement the functions of the innovations herein. Or, the modules can comprise programming instructions transmitted to a general-purpose computer or to processing/graphics hardware via a transmission carrier wave. Also, the modules can be implemented as hardware logic circuitry implementing the functions encompassed by the innovations herein. Finally, the modules can be implemented using special purpose instructions (SIMD instructions), field programmable logic arrays or any mix thereof which provides the desired level performance and cost.

As disclosed herein, features consistent with the disclosure may be implemented via computer-hardware, software, and/or firmware. For example, the systems and methods disclosed herein may be embodied in various forms including, for example, a data processor, such as a computer that also includes a database, digital electronic circuitry, firmware, software, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the invention or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the invention, or it may be more convenient to construct a specialized apparatus or system to perform the required methods and techniques.

Aspects of the method and system described herein, such as the logic, may also be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) though again does not include transitory media. Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

Although certain presently preferred implementations of the invention have been specifically described herein, it will be apparent to those skilled in the art to which the invention pertains that variations and modifications of the various implementations shown and described herein may be made without departing from the spirit and scope of the invention. Accordingly, it is intended that the invention be limited only to the extent required by the applicable rules of law.

While the foregoing has been with reference to a particular embodiment of the disclosure, it will be appreciated by those skilled in the art that changes in this embodiment may be made without departing from the principles and spirit of the disclosure, the scope of which is defined by the appended claims.

What is claimed is:

1. A device, comprising:
a first leg element made by three dimensional printing, the first leg element having a first end and a second end at each end of a longitudinal member;
a first loop attached adjacent the first end of the first leg element;
a second loop attached adjacent the second end of the first leg element; and
the first and second loops made by three dimensional printing.

2. The device of claim 1, wherein the first leg element has a cylindrical shape.

3. The device of claim 1, wherein the first leg element and the first and second loops are each made of a photo-curable polymer.

4. The device of claim 3 further comprising a drug embedded in the first leg element and the first and second loops.

5. The device of claim 1, wherein the first leg element and the first and second loops are each made of a composite of a polymer and a plurality of micro-nanoparticles.

6. The device of claim 1, wherein the first leg element and the first and second loops are each made of a mixture of conductive materials.

7. The device of claim 1, wherein the first leg element and the first and second loops are each made of a poly urethane shape memory polymer.

8. The device of claim 1 further comprising a second device made with three dimensional printing having a second device first leg element having a first end and a second end at each end of a longitudinal member, a first loop of the second device attached adjacent the first end of the first leg element and a second loop of the second device attached adjacent the second end of the first leg element and wherein the device and the second device are connected to each other.

9. The device of claim 8, wherein the device and the second device slide relative to each other when assembled.

10. The device of claim 8, wherein the device and the second device are connected in a line with each other.

11. The device of claim 8, wherein a plurality of devices are connected in pairs to each other.

12. The device of claim 8, wherein a plurality of devices are connected together in a circuit.

13. A device, comprising:
a first leg element made by three dimensional printing, the first leg element having a first end and a second end at each end of a longitudinal member;
a second leg element made by three dimensional printing, the second leg element having a first end and a second end at each end of a longitudinal member, wherein the first ends of the first and second leg elements are connected together and the second ends of the first and second leg elements are connected together;
a first loop attached adjacent to the first end of the first leg element;
a second loop attached adjacent to the second end of the second leg element; and
the first and second loops made by three dimensional printing.

14. The device of claim 13, wherein the first and second leg elements each have a cylindrical shape.

15. The device of claim 13, wherein the first and second leg elements and the first and second loops are each made of a photo-curable polymer.

16. The device of claim 15 further comprising a drug embedded in the first leg element and the first and second loops.

17. The device of claim 13, wherein the first and second leg elements and the first and second loops are each made of a composite of a polymer and a plurality of micro-nanoparticles.

18. The device of claim 13, wherein the first and second leg elements and the first and second loops are each made of a mixture of conductive materials.

19. The device of claim 13, wherein the first and second leg elements and the first and second loops are each made of a poly urethane shape memory polymer.

20. The device of claim 13 further comprising a second device made with three dimensional printing having a second device first and second leg elements each having a first end and a second end at each end of a longitudinal member, a first loop of the second device attached adjacent the first end of the first leg element and a second loop of the second device attached adjacent the second end of the first leg element and wherein the device and the second device are connected to each other.

21. The device of claim 20, wherein the device and the second device slide relative to each other when assembled.

22. The device of claim 20, wherein the device and the second device are connected in a line with each other.

23. The device of claim 20, wherein a plurality of devices are connected in pairs to each other.

24. The device of claim 20, wherein a plurality of devices are connected together in a circuit.
